(19) United States
(12) Patent Application Publication
(10) Pub. No.: US 2021/0161423 A1
(43) Pub. Date: Jun. 3, 2021

(54) Apparatus and Method for Optical Spectroscopy and Bioimpedance Spectroscopy using a Mobile Device Case to Gather Physiological Information

(71) Applicant: Vita Analytics Inc., Cupertino, CA (US)

(72) Inventors: Aaron Olbrich, Morgan Hill, CA (US); Bijoy Purushothaman, Westminister, CO (US); Manish Dalwani, Westminister, CO (US); Sachin Ramesh Gandhi, San Jose, CA (US)

(73) Assignee: Vita Analytics Inc., Cupertino, CA (US)

(21) Appl. No.: 15/774,965

(22) PCT Filed: Feb. 21, 2018
(86) PCT No.: PCT/US18/19014

§ 371 Date: May 9, 2018

Related U.S. Application Data
(60) Provisional application No. 62/580,286, filed on Nov. 1, 2017.
(60) Provisional application No. 62/461,666, filed on Feb. 21, 2017.

(51) Int. Cl. A61B 5/0536; A61B 5/145; A61B 5/00

(52) U.S. Cl. CPC A61B 5/0536; A61B 5/14532; A61B 2560/0468; A61B 5/6898; A61B 5/6826

(57) ABSTRACT

A mobile touchable system for user fitness and health monitoring is presented. The system is designed in one form as a mobile phone case and in another form as integrated in a mobile phone. It encompasses a series of measurement devices, including, but not limited to, arrays of electrodes for bio-impedance analysis, impedance tomography, and electrocardiographs, near-infrared spectroscopy for glucose level measurement, and heart rate monitoring. The touchable system performs incidental measurement in the background each time the user holds the mobile phone and hence enables long term health monitoring without user intervention. The touchable monitoring system further performs targeted spot measurements by following defined procedures. Spot measurement is enhanced through an extension measurement cable. Furthermore, an extension strap along with the mobile touchable system provides detailed activity tracking. The touchable system performs long term health monitoring, and provides early alerts for abnormal condition such as diabetes, dehydration, hypertension, cardiovascular anomalies, breast cancer and prostate cancer.

### CROSS REFERENCE TO RELATED APPLICATIONS

This application claims priority under 35 USC 371 to International Application PCT/US18/19014, filed on Feb. 21, 2018, entitled, “Apparatus and Method for Optical Spectroscopy and Bioimpedance Spectroscopy using a Mobile Device Case to Gather Physiological Information,” which is incorporated by reference in its entirety. PCT/US18/19014 claims priority to U.S. provisional patent application No. 62/580,286 filed Nov. 1, 2017 entitled, “Apparatus and Method for Optical Spectroscopy using a Phone Case to Gather Physiological Information,” which is hereby incorporated by reference in its entirety. PCT/US18/19014 further claims priority to U.S. provisional patent application No. 62/461,666 filed Feb. 21, 2017 entitled, “Apparatus and Method for Mobile Activity and Health Monitoring System through Incidental Touch of Mobile Phone,” which is also hereby incorporated by reference in its entirety.

### TECHNICAL FIELD

The present application relates to mobile touchable system for physiological fitness and health monitoring.

### BACKGROUND

Health monitoring involves expensive, cumbersome and even painful test procedures. As a result, the majority of the population has sparse and scarce data points about their own body and personal health. For instance, even with breast cancer risk on the rise, a mammogram may be performed only once every 24 months. On the other hand, early detection vastly improves a patient's prognosis. In most cases, people on the verge of diabetes or hypertension (i.e. people with prediabetes or prehypertension) go to a clinic or a medical facility once a year and thus the clinical information is episodic. Daily information on glucose or blood pressure can stop or delay the onset of diagnostic diabetes or hypertension respectively. Most diabetic patients rely on taking blood samples by finger pricking using lancets for glucose level measurements, which again are limited samples and the repetitive procedure itself can be painful.

A number of wearable activity monitoring devices are available on the market, most prominently Apple Watch and a series of FitBit devices. They provide the benefit of continuous measurement. However, these devices mostly record a spot measurement of basic parameters such as heart rate, motion etc. and provide very little insight into the general health of a user. Furthermore, these wearable devices suffer from fading consumer interest because they require skin contact with the wrist and become uncomfortable. On the other hand, the mobile phone has increasingly become an indispensable part of every person's life. A study shows that an average user holds 150 mobile phone sessions each day. The mobile phone is already the most touched device.

There are a wide range of non-intrusive procedures popular in both clinical settings and, more recently, the fitness industry. Bioimpedance analysis is one such procedure. Bioelectrical spectroscopy (BIS) uses mathematical modeling and mixture equations to determine body fat, fat-free mass (FFM), total body water (TBW) consisting of extra-cellular water (ECW) and intra-cellular water (ICW). Body hydration management is a key objective in the sports and fitness world, and has a positive impact on mental functioning and general physiological health maintenance. The ratio of ECW/ICW is a key indicator of tissue health including general inflammation levels. On the other hand, the percentage body fat has a direct impact on the health condition and is in most times a key indicator for several pathophysiological conditions, including breast cancer and prostate cancer.

Other popular non-intrusive procedures include electrocardiogram (EKG), generally performed to treat a clinical condition, that is critical for monitoring information about the structure and function of the heart. Near infrared spectroscopy is another important example of a measurement procedure for blood glucose level monitoring.

Health measurement and monitoring of a clinical condition often involves expensive tests, and there are sparse and scarce data points. On the other hand, current activity tracking systems, most notably wearable products, suffer from over simplistic measurement and improper sensor contact location, mostly involving only heart rate and motion sensing.

### SUMMARY

An apparatus and method for optical spectroscopy and bioimpedance spectroscopy using a mobile device case to gather physiological information is disclosed. According to one embodiment, a system comprises a case suitable for use with a mobile device; and a first recess in the case. The first recess has first optoelectronic sensors and first electrodes that facilitate one or more scans. The scans include bioimpedance measurements and optical scans.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views.

FIG. 1 depicts a touchable health and activity monitoring system with a mobile phone case, according to one embodiment.

FIG. 2 depicts optical spectroscopy performed with the mobile phone case, according to one embodiment.

FIG. 3 depicts components of a mobile phone that trigger automatic measurements, according to one embodiment.

FIG. 3 depicts measurement through an incidental touch performed with the mobile phone case, according to one embodiment.

FIG. 4 depicts a system for collecting health and activity data collected by mobile phone case, according to one embodiment.

FIG. 5 depicts a system that measures vital body parameter trends over time, using analytics within a mobile device or in the cloud, according to one embodiment.

FIG. 6 depicts a touchable mobile phone with a circular array of electrodes, according to one embodiment.

FIG. 7 depicts the mobile phone case with a matrix array of electrodes, according to one embodiment.

FIG. 8 depicts the mobile phone case with an electrode extension cable, according to one embodiment.

FIG. 9 depicts a user making a bioimpedance measurement using an electrode extension cable attached to the mobile phone case, according to one embodiment.

FIG. 10 depicts a touchable mobile phone case strap design wrapped around a user's arm, according to one embodiment.

FIG. 11 depicts an expanded view of a mobile phone case having a finger notch sensor for improved haptic feedback to the user, according to one embodiment.

FIG. 12 depicts an optical scan apparatus in the mobile phone case being used to study the optical response characteristics of fruits, vegetables and other food substances to determine freshness or levels of decay, according to one embodiment.

FIG. 13 is a block diagram of an exemplary spectrometry circuit, according to one embodiment.

FIG. 14 depicts the mechanical integration of electronics platform and the phone case, according to one embodiment.

FIG. 15 illustrates a process for data collection by incidental touch, according to one embodiment.

FIG. 16 illustrates a process for spot measurement, according to one embodiment.

FIG. 17 illustrates a process for segment measurement, according to one embodiment.

FIG. 18 illustrates a process for deep data analysis using the present mobile phone case, according to one embodiment.

FIG. 19 illustrates a process for collecting data for haptic feedback, according to one embodiment.

### DETAILED DESCRIPTION

The touchable phone case design provides sampled measurement of multiple vital body parameters. This enables a big data approach for the user health condition. A single user is analyzed over time for individualized trend analysis. Regular (whether periodic or random) monitoring and feedback allows users to make micro adjustments in behavior on daily basis and macro adjustments of habits over longer periods. These include behaviors and habits in diet, hydration, exercise, and supplements among others. For healthcare patients, body parameters before, during and after the administration of medicine allows for detailed and individualized evaluation of medication effects. Furthermore, statistical analysis can be applied to groups of users with similar backgrounds. Such backgrounds may include similar age, gender, ethnic group and family history. Cross-comparison of the data can lead to insight into individual health risk, and serves as an early alert for abnormal health condition.

For all types of measurements, along with accuracy, reliability is equally important. The large volume of data that the touchable phone case collects helps improve both consistency and precision. Furthermore, not every user touch or measurement may lead to a successful measurement, and accordingly, machine learning is applied to filter out bad measurements as noise. Given the daily data collection, statistical analysis will be applied to detect short- and long-term trend changes in various health care biometrics measures.

The following disclosure provides many different embodiments, or examples, for implementing different features of the subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

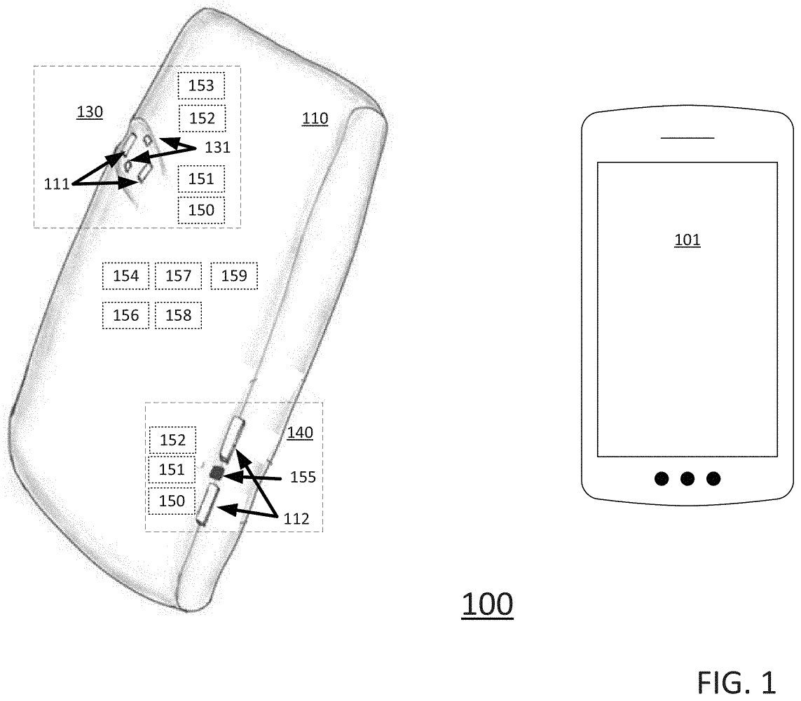

FIG. 1 depicts a touchable health and activity monitoring system with a mobile phone case, according to one embodiment. Health and activity monitoring system 100 includes a mobile phone case 110 and a mobile phone or device 101 that may be used for collecting and analyzing health and activity data from a user. With minimal or no user interaction, the mobile phone case 110 collects health and activity data when the user makes contact with the sensors on the case, such as when the user picks up the case. The two main sensor locations are on a side of the case, allowing for the user's finger to rest on a groove-like, finger notch sensor 130. The finger notch sensor 130 houses optical spectroscopy sensors 131 along with electrodes 111. An additional sensor 140, such as a bioimpedance sensor, also houses electrodes 112, and is located on the diagonally opposite side of the case from finger notch sensor 130, where a groove allows for a user's palm to contact the mobile phone case 110. These positions may be reversed for users who hold the mobile phone case 110 in their right hand. In another embodiment, the finger notch that houses the optical spectroscopy sensors may be moved to the center of the case to make a common design for both right- and left-handed users.

Sensors 130 and 140 may include a photodiode array 150, an LED array 151, and a laser diode array 152. The photodiode array 150 may be placed substantially orthogonal to the LED array 151 or laser diode array 152, according to one embodiment.

In certain embodiments, the mobile phone case 110 contains additional types of sensors such as pressure sensors 153 attached to electrodes 111 and 112, moisture sensors 154, temperature sensors 155, and humidity sensors 156, built in to the phone case to aid, for example, in the monitoring of measurement quality.

In addition to the sensors and accessories described above, the following additional sensors may be used with the present system:

GSR sensor 157 measures galvanic skin resistance to calculate skin moisture and conductivity. This data may also be used to measure stress.

Infrared thermopile sensor 158 is used for contactless temperature measurement.

Sweat analyzer 159 is a sensor that analyzes sweat to detect dehydration as well as various types of diseases.

In one embodiment, the LED array 151 may be deployed as a light source and the laser diode array 152 may be deployed as a detector. Multiple LEDs of various center wavelengths can be produced to generate a light source to perform a scan over a large range of frequencies. According to one embodiment, the touchable mobile phone case 110 may contain an array of four (4) LEDs as sources. The number of LEDs in the array could be larger, in one embodiment, with eight (8) or even twelve (12) LEDs that cover a wide spectrum of wavelengths (ranging from visible to mid-infrared regions).

The LED array 151 and the laser diode array 152 may be deployed in multiple arrangements applying transmission/reflection/refraction spectroscopy. According to one embodiment, they may be arranged across from each other for measurement of light transmitted through the user's tissue. In an orthogonal arrangement, the laser diode array 152 measures light generated by the LED array 151 that is scattered or diffracted through the tissue. In a side by side arrangement the laser diode array 152 measures light generated by LED array 151 reflected from the user's tissue.

In another embodiment, the LED array 151 could be deployed in a tightly packed package right at the source placement. According to another embodiment, the LEDs could be placed at the convenient location where spacing is less of a limitation to expand the number of LEDs with light carried to the source placement using optic fibers or light pipes. In another embodiment, the mobile phone case 110 can include arrays of electrodes for bioimpedance spectroscopy, LEDs for near infrared spectroscopy, and LEDs for heart rate monitoring.

In another embodiment, the mobile phone case 110 implements optical scanning via optical spectroscopy in the visible and near infrared regions using the integrated LED array 151 and the laser diode array 152. According to another embodiment, the laser diode array 152 performs scattering spectroscopy (Raman Scattering) using light in visible to infrared regions.

In an alternate embodiment, sensors contained in mobile phone case 110, can be integrated directly into the mobile phone 101 and function in the same manner. The foregoing description uses the example of mobile phone case 110 for simplicity of explanation of the present system.

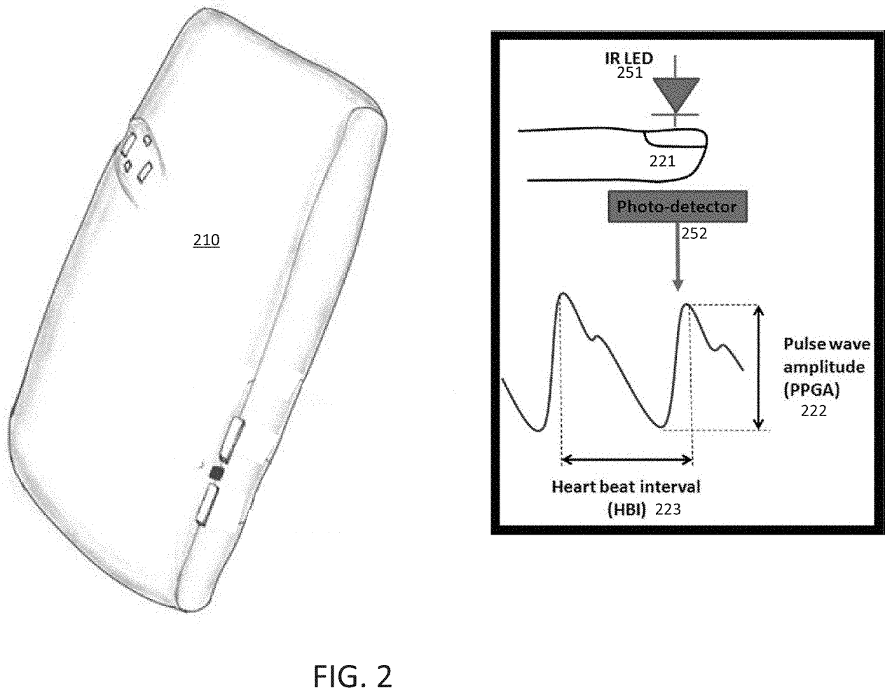

FIG. 2 depicts optical spectroscopy performed with the mobile phone case, according to one embodiment Mobile phone case 210 performs optical spectroscopy using the integrated LED array 251 as an IR source, and the laser diode array 252 as the photodetector. To capture a Photoplethysmography (PPG) signal 222 from the user's fingertip 221, light generated by integrated LED array 251 passes from one side of the finger 221 and is captured on the other side by the laser diode array 252. The output of the analysis is a waveform that corresponds to the pulse wave amplitude (“PPGA”) 222 and heartbeat interval (“HBI”) 223. Various characteristics of the PPGA signal HBI signal are analyzed using a process, such as Beer-Lambert law to gather additional information about the user's health.

The measurements described above can be performed without any human intervention, and they provide continuous measurement data for the human body, called incidental measurements.

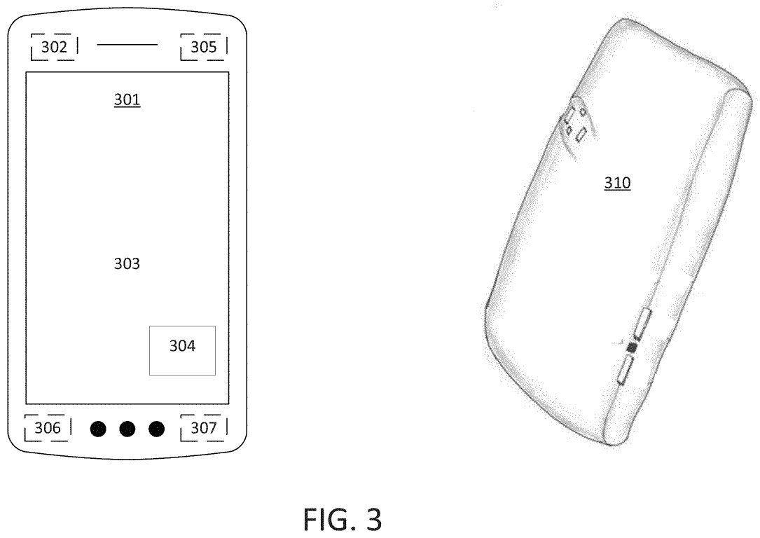

FIG. 3 depicts various components of a mobile phone that trigger automatic measurements, according to one embodiment. First, there are built in sensors 302 in most modern mobile phones 301 to detect rotation change and wake up the screen 303, triggering the mobile app 304 to launch and take a measurement attempt at the same time. Second, built-in pressure sensors 305 can trigger the mobile app to make another measurement attempt. In another embodiment, measurement can be triggered by capacitive sensing enabled through the metal electrodes 306 on the mobile phone 301. Capacitive sensors 307 can also be placed under the surface of the phone covering in close proximity, to detect user contact, and trigger incidental measurements. For example, a single touch by a user on the screen 303 having capacitive sensors 307 can initiate a scan by activating the bioimpedance sensors or any of the sensors described above. According to one embodiment, the material of the phone case acts as a dielectric medium. According to one embodiment, a user would use her finger to tap over the screen 303 having capacitive touch sensors 307. In another embodiment capacitive touch sensors are within the finger notch, such that a user can tap her finger in the finger notch to activate the system intentionally. Depending on the number of taps, different aspects of the measurement system could be activated, according to one embodiment.

The type of incidental measurement depicted in FIG. 3 is built upon existing user habit with a mobile phone, and it does not alter user behavior in any aspect. As such, it enables regular measurements throughout the day, and thereafter statistical analysis using deep data mining techniques. This represents a complete paradigm shift from current state-of-the-art health monitoring, where measurement data is sparse and scarce.

In an alternate embodiment, bioimpedance analysis (“BIA”) for biometric identification is performed by mobile phone case 310. The system utilizes user-specific unique impedance response patterns to electrical stimulation to determine user signatures allowing for identification of a user. The present system can use Bluetooth and/or WiFi communication mechanisms to interact with external systems to employ BIA-based biometric identification. External systems may be a mobile device within the case, or other computing devices within range of the case 310. The system, according to one embodiment, performs BIA measurements for many types of BIA applications.

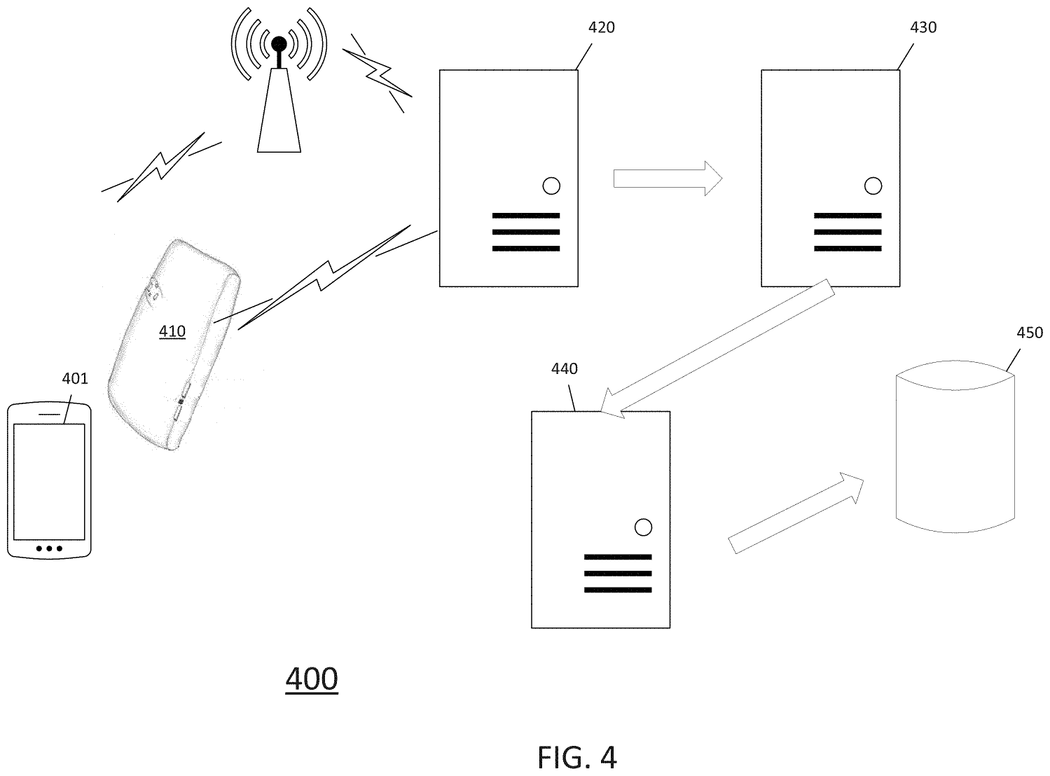

FIG. 4 depicts a system for collecting health and activity data from the present mobile phone case, according to one embodiment. FIG. 4 depicts a system 400 where the health and activity data collected by mobile phone case 410 can be transmitted to a centralized server 420 for various processing and analytics, according to one embodiment. The centralized server 420 may transfer data with an analytics server 430, such as a cloud server, or an enterprise server. Each mobile phone device 401 or mobile phone case 410 that is deployed can connect to the centralized server 420 via a WiFi connection 421 or via cellular network system 422.

In another embodiment, health and activity data collected from the mobile device case 410 is transmitted to the centralized server 420, and then transferred to and stored on analytics server 430 for the purpose of performing data analytics. As data is collected from a sufficiently large sample set of users, deep data mining (e.g., impedance body fat, ICW, ECW, reactance, phase, tomographic images, etc.) may be performed to establish an individualized baseline for a user. According to one embodiment, BIA measurements are performed using sinusoidal signals in the 10 kHz-1 MHz range. Furthermore, the same electrodes that are used for BIA can be repurposed into a listening mode to function as ECG electrodes. The analytics server 430 may monitor statistics to detect shifts from the user's baseline and interpret anomalies to infer health conditions. A communications server 440 allows for comparison of individual user data to a larger population provides for deeper analysis and allows for better diagnosis of health conditions.

In another embodiment, the communications server 440 provides anonymous user social networking to enable communications between users with similar physiologies and pathologies. According to another embodiment, the system provides a portal 450 for healthcare infrastructure for primary care physicians to monitor users offline. Individual users may choose to release data for research studies on an anonymous basis.

In another embodiment, the mobile phone case 410 may be used for various applications, such as glucose monitoring, blood pressure monitoring, heart rate monitoring, alcohol level monitoring and testing, and testing for other specific substances in blood stream including molecules released into blood stream from medications. Data collected from these applications can be provided to centralized server 420 for processing, and analytics server 430 for data analytics, according to one embodiment.

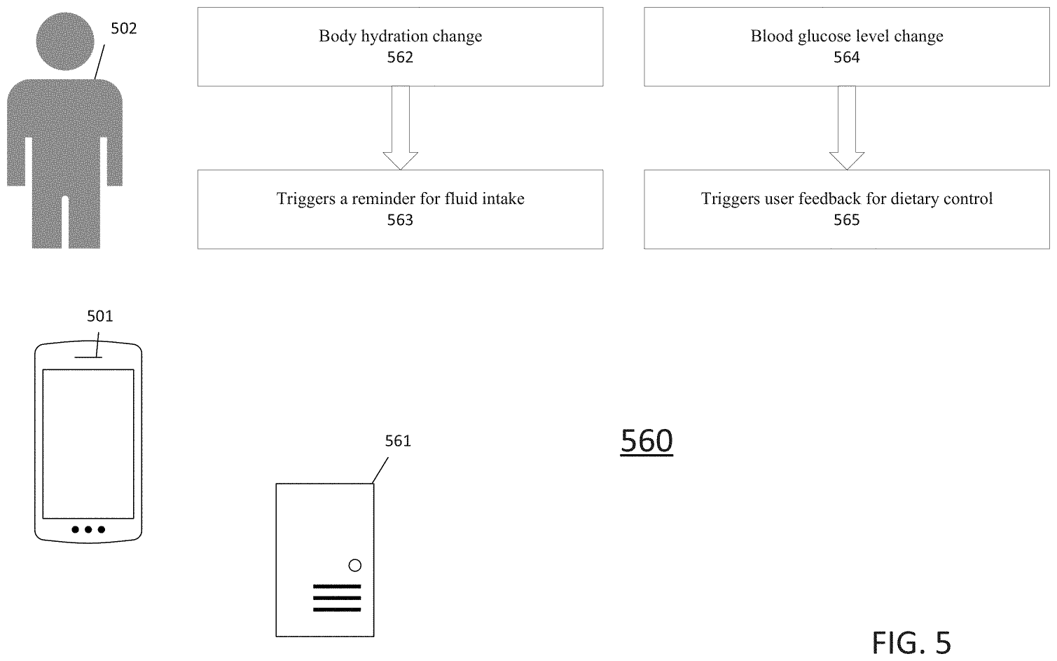

FIG. 5 depicts a system that measures vital body parameter trends over time, using analytics within a mobile device or in the cloud, according to one embodiment. As depicted in FIG. 5, a system 560 allows for measuring vital body parameter trends over time using analytics within a mobile device 501 or in the cloud 561. This analysis and instant feedback allows the user 502 to adjust activity behavior at granular level. Body hydration change 562, for instance, can be used to trigger a reminder for fluid intake 563. A blood glucose level change 564 will trigger user feedback for dietary control 565, including changing both type of food consumption and timing of food intake. Such behavior changes provide benefits to maintain and improve the body's health, and can also aid in treating certain users with health ailments, such as diabetes. Spot measurement data can be used in an analytics platform to establish a normal baseline for user 502, and further for trend analysis to monitor for aberrations and anomalies.

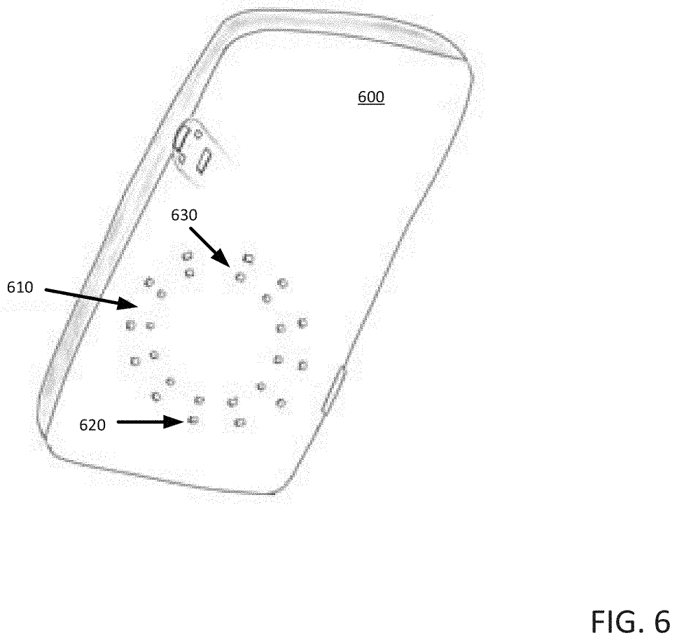

FIG. 6 depicts a touchable mobile phone with a circular array of electrodes, according to one embodiment. FIG. 6 depicts the mobile phone case 600 with a circular array of electrodes 610 on the back of the case. The circular array 610 is used for bioimpedance measurement with active electrodes 620 and passive electrodes 630 similar to a 4 electrode tetra polar electrode arrangement (i.e. 2 voltage and 2 current electrodes) being made by electrical multiplexing. BIA in this setup may be performed with the tetra polar set up (with electrodes on the side of the case) in a frequency range of 10 KHz-1 MHz and the frequency range may be expanded beyond 1 MHz to explore tissue/bone properties in finer detail.

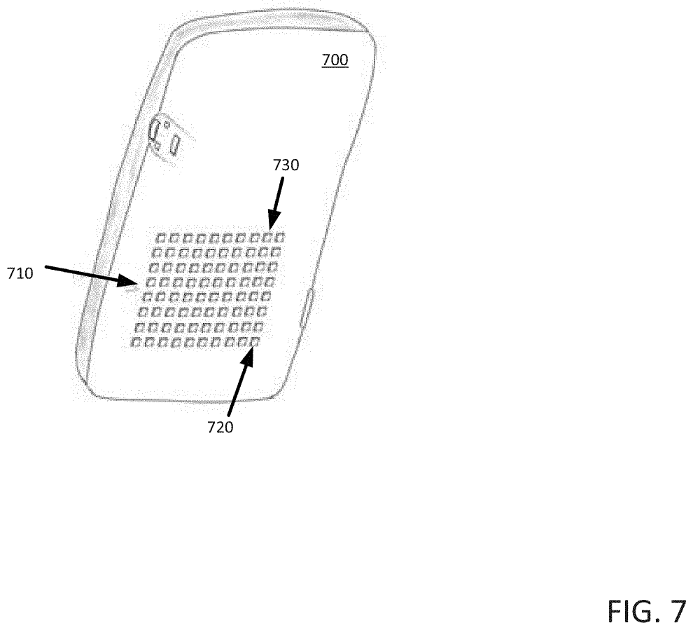

FIG. 7 depicts the mobile phone case with a matrix array of electrodes, according to one embodiment. FIG. 7 depicts the mobile phone case 700 with a matrix array of electrodes 710 on the back of the case. The matrix array 710 is used for bioimpedance measurement with active electrodes 720 and passive electrodes 730 similar to standard tetra polar electrode arrangement being made by electrical multiplexing. A similar BIA frequency range (e.g., 10 KHz-1 MHz or beyond) may be applied to this situation of matrix array of electrodes.

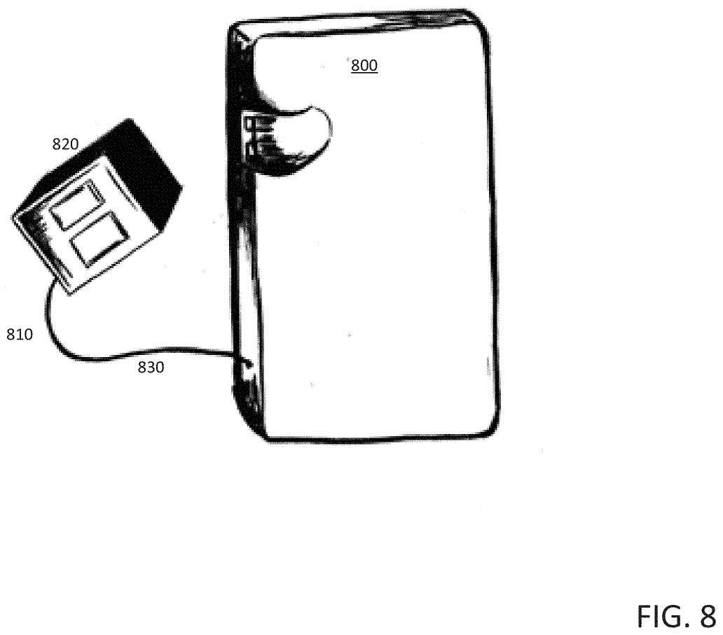

FIG. 8 depicts the mobile phone case with an electrode extension cable, according to one embodiment. FIG. 8 depicts the mobile phone case 800 with an electrode extension cable 810, connecting to bioimpedance extension module 820. The extension cable 810 contains a set of electrodes 830 used to make bioimpedance measurements across the body, for example the front and back of the user's trunk. A similar BIA frequency range (e.g., 10 KHz-1 MHz or beyond) may be applied to this embodiment.

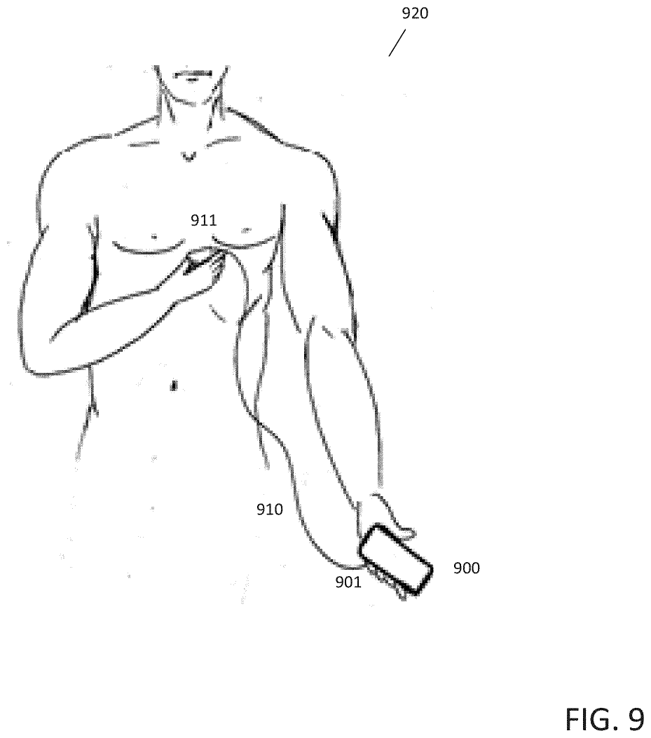

FIG. 9 depicts a user making a bioimpedance measurement using an electrode extension cable attached to the mobile phone case, according to one embodiment. FIG. 9 depicts a user 920 making a bioimpedance measurement using an electrode extension cable 910 attached to mobile phone case 900. In one embodiment, the electrode extension cable 910 accessory allows for a focused spot measurement to be recorded. The electrode extension cable 910 can be plugged into the mobile phone case 900 through the data port 901. The extension cable 910 can be equipped with additional electrodes 611 at one end. Upon detecting the extension cable 910, a mobile app prompts the user for a targeted measurement, for instance, bioimpedance by touching the mobile phone case 900 or extension cable 910 to the user's breast issue. In another example, a user may perform BIS through the liver by holding the mobile phone case 900 or extension cable 911 against the abdomen and extending the electrodes 911 to his lower back.

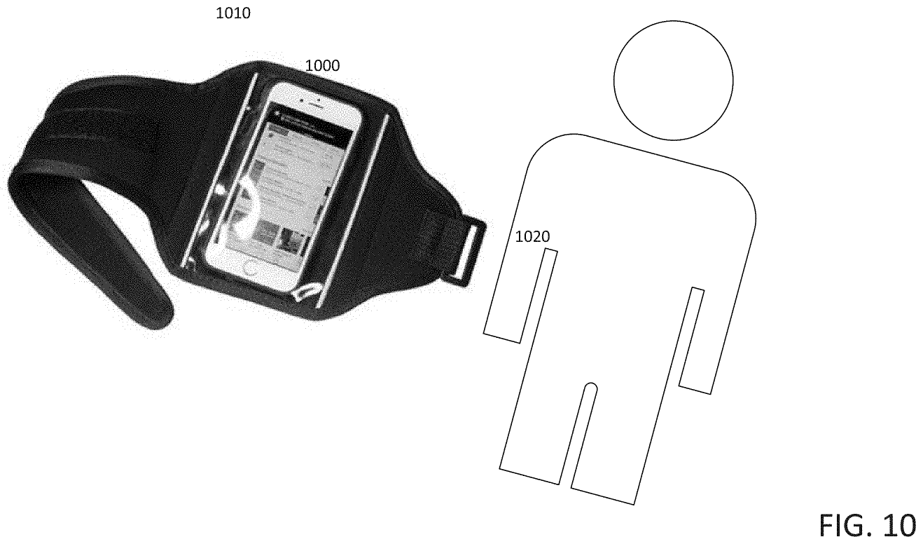

FIG. 10 depicts a touchable mobile phone case strap design wrapped around a user's arm, according to one embodiment. FIG. 10 depicts a touchable mobile phone case strap design 1010 wrapped around a user's arm 1020. The mobile phone case strap design 1010 contains the touchable mobile phone case 1000, and may be wrapped around the user's arm 1020. When a user wears the touchable mobile phone case 1000 on the arm 1020 during exercise, the phone case is in touch with skin, and can measure continuous user data such as heart rate, glucose level, blood oxygen level, and hydration level. Monitoring such continuous data, and allowing for immediate feedback, allows users to achieve the maximum benefit of a workout, for example. A similar BIA frequency range (e.g., 10 KHz-1 MHz or beyond) may be applied to this embodiment.

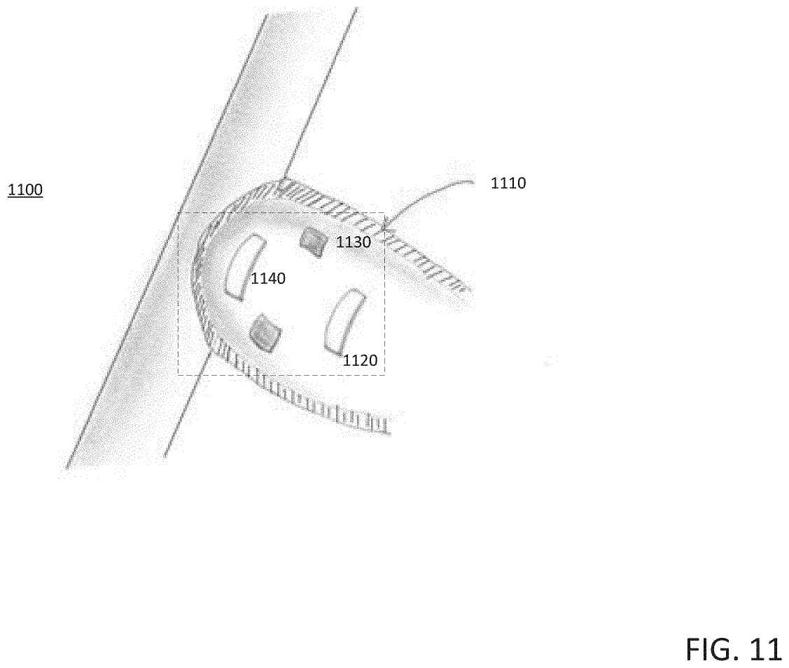

FIG. 11 depicts an expanded view of a mobile phone case having a finger notch sensor for improved haptic feedback to the user, according to one embodiment. The present case may include a softer material around the finger notch 1110 laid on to the finger notch sensor 1120 for improved haptic feedback to the user. The softer material can also be applied to the sensor 1130, where the user's palm contacts the case. In certain embodiments, the mobile phone case 1100 has tactile sensors 1140 that measure forces exerted by the user on the interface, such as at finger notch sensor 1120 and palm sensor 1130. The softer material allows for increased sensitivity to user touch and improved haptic feedback upon contact with the user's finger or palm. Tactile sensors 1140 also guide the user to properly position her finger in contact with the finger notch 1110. For example, the user may feel a buzz when her finger is properly placed within the finger notch 1110.

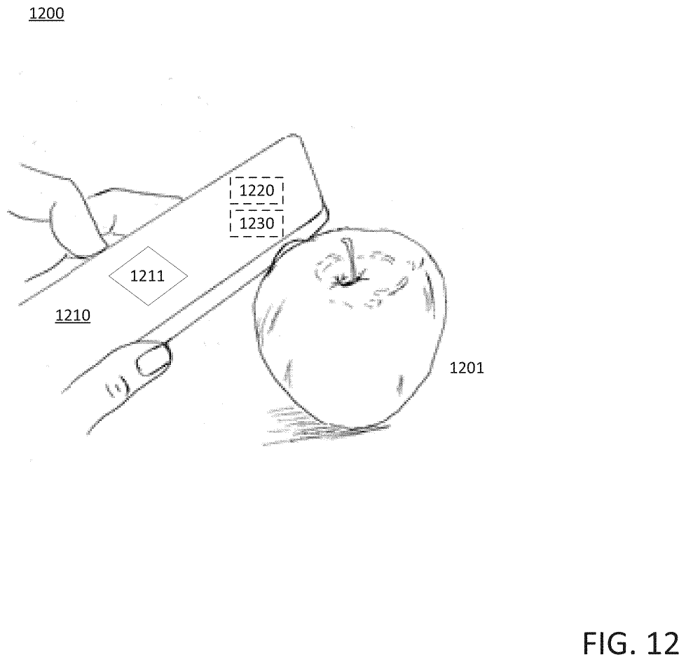

FIG. 12 depicts an optical scan apparatus in the mobile phone case being used to study the optical response characteristics of fruits, vegetables and other food substances to determine freshness or levels of decay, according to one embodiment. FIG. 12 depicts an optical scan apparatus 1200 in the mobile phone case 1210 being used to study the optical response characteristics of fruits, vegetables and other food substances 1201 to determine freshness or levels of decay. Optical sensors 1220 located on an outer side of the phone case, include an array of photodetectors 1230, and the user can place the phone case near a food substance 1201 for evaluation. The photodetectors 1230 can determine the freshness of a given food substance based on comparing measured data with known data characteristics of that substance, such as coloring or transparency, which can indicate whether the food substance, for example, is fresh. The system 1200 uses absorbance principles to look for the presence of certain materials that indicate decay. This comparison and evaluation process can be driven by a mobile app installed on the mobile phone 1211.

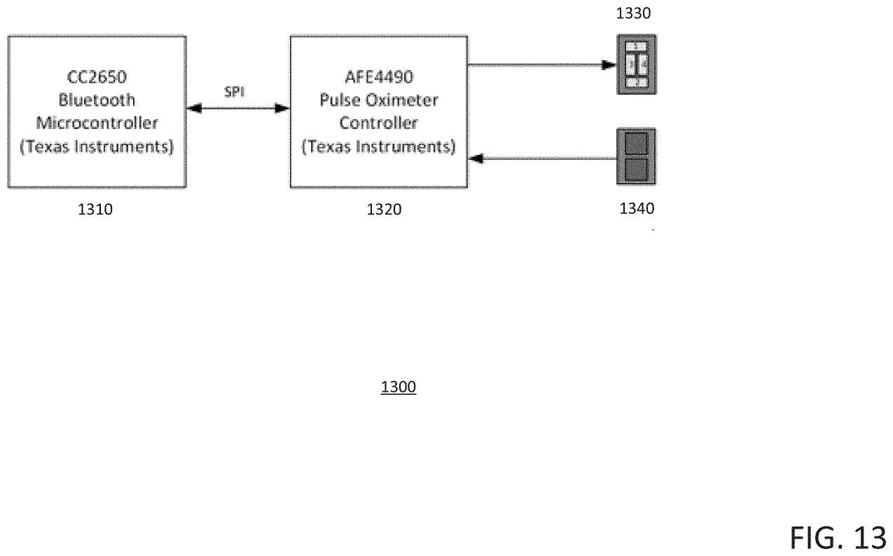

FIG. 13 is a block diagram of an exemplary spectrometry circuit, according to one embodiment. The spectrometry circuit 1300 includes a Bluetooth microcontroller 1310, such as a CC2650 chip from Texas Instruments. The spectrometry circuit also includes a pulse oximeter controller 1320, such as the AFE4490 chip from Texas Instruments. The two chips communicate with each other using the SPI protocol. The spectrometry circuit also includes an LED array where there are at least four (4) LEDs 1330. According to one embodiment, the LEDs may be one or more of a 640 nm Red LED (MTPS9067MC), 940 nm IR LED (MTE9460MC), 1200 nm IR LED (MTSM0012-843-IR), or 1550 nm IR LED (MTSM0012-843-IR). These LEDs may be obtained from Marktech Optoelectronics with the model information provided above, or from any other suitable LED manufacturer. The photo diode array 1340 may be one or more of the MTPD1346D-100 from Marktech Optoelectronics or TEMD7100X01 from Vishay Semiconductor, according to one embodiment. According to one embodiment, the power of the LED array may be controlled to improve the signal quality for each user of the case.

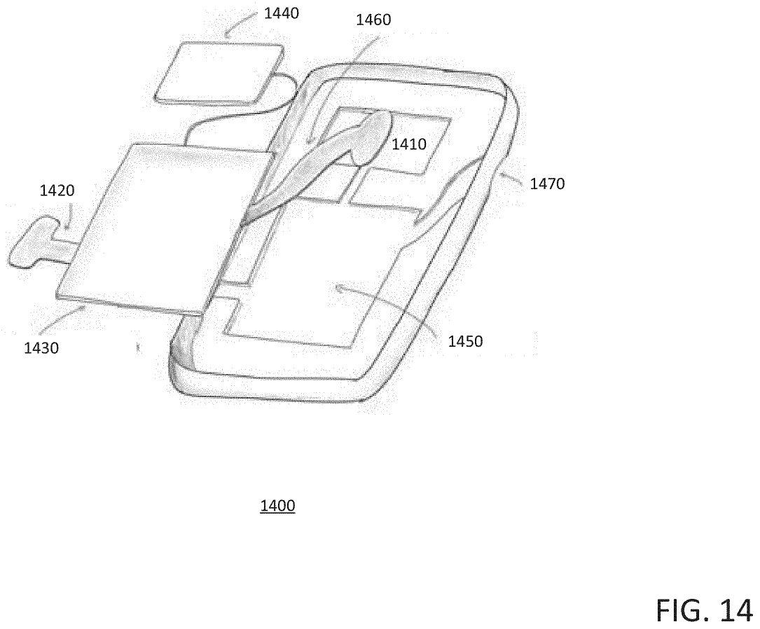

FIG. 14 depicts the mechanical integration of electronics platform and the phone case 1400, according to one embodiment. Sensors, such as a flex to palm electrodes 1410, are attached to flex cables 1420 that are built into the main PCB 1430. In certain embodiments, a power source such as a battery 1440 is connected to main PCB 1430. The battery 1440 and main PCB 1430 fit into cutouts in the phone case body 1450, allowing for the flex finger notch sensor 1460 to protrude through to the other side of the case to the finger notch position 1470.

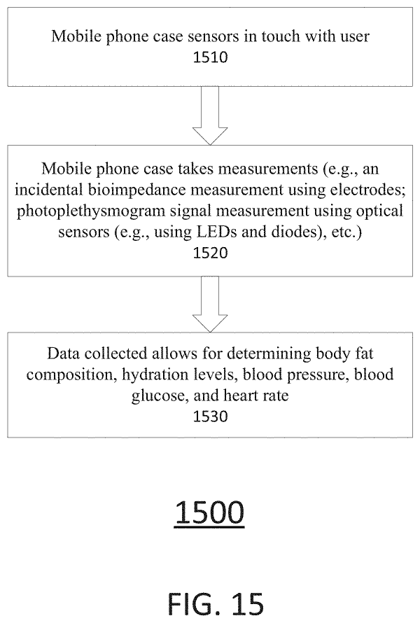

FIG. 15 illustrates a process for data collection by incidental touch, according to one embodiment. Additional measurements can be performed with the mobile phone case 1500. As depicted in FIG. 15, one type of measurement is achieved through incidental touch, for example, when a user touches the mobile phone case at 1510. Users typically make such touches with mobile phones more than 150 times each day. At each touch, the mobile phone case can perform incidental bioimpedance measurements using electrodes located at both sides of the phone case or incidental optical PPG measurements using LEDs and Photodiode sensors in the finger notch at 1520. The data collected allows for determining body fat composition, hydration levels, blood glucose, blood pressure, blood oxygen saturation and heart rate at 1530.

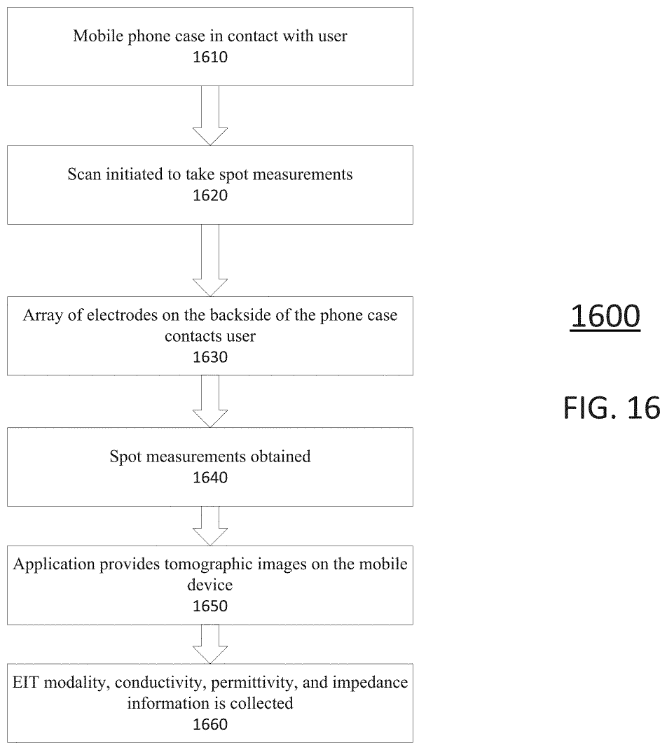

FIG. 16 illustrates a process for spot measurement, according to one embodiment. As depicted in FIG. 16, the mobile phone case can be used to take spot measurements, occurring when the user intentionally takes measurements by holding the mobile phone case 1600 in a recommended manner at 1610. The user then starts a scan at 1620 by either triggering it from the mobile app or by depressing a switch on the phone case. According to one embodiment, the user obtains spot measurements at 1640 by holding the phone against a targeted body position, making contact with the functional array of electrodes on the backside of the phone case at 1630, which provides the user with tomographic images or detailed spectroscopic information of the scanned tissue (e.g., tissue/organ specific spectroscopic information, local cell health, etc.) on the mobile app at 1650. The mobile app can accomplish this through electrical impedance tomography (“EIT”). The array of electrodes serve as a closed domain under this test. Electrical current is sent through a pair of driving electrodes and the voltage information is collected from a pair of sensing electrodes. The process is repeated until each electrode pair has served as a driving electrode while the other electrode pair automatically takes the role of a sensing electrode. EIT modality, conductivity, permittivity, and impedance information is collected at 1660 from the surface electrodes on the backside of the phone case. In another embodiment, an additional accessory, having the same or greater number of electrodes than the number of electrodes on the phone case can provide for a higher resolution tomographic images. The images obtained by spot measurements can provide important electrical property data electrical properties vary from organ to organ, as well as from normal tissue versus abnormal tissue. Studies have shown that abnormal electrical property data can give an early indication of breast cancer, liver disease and prostate cancer, for example.

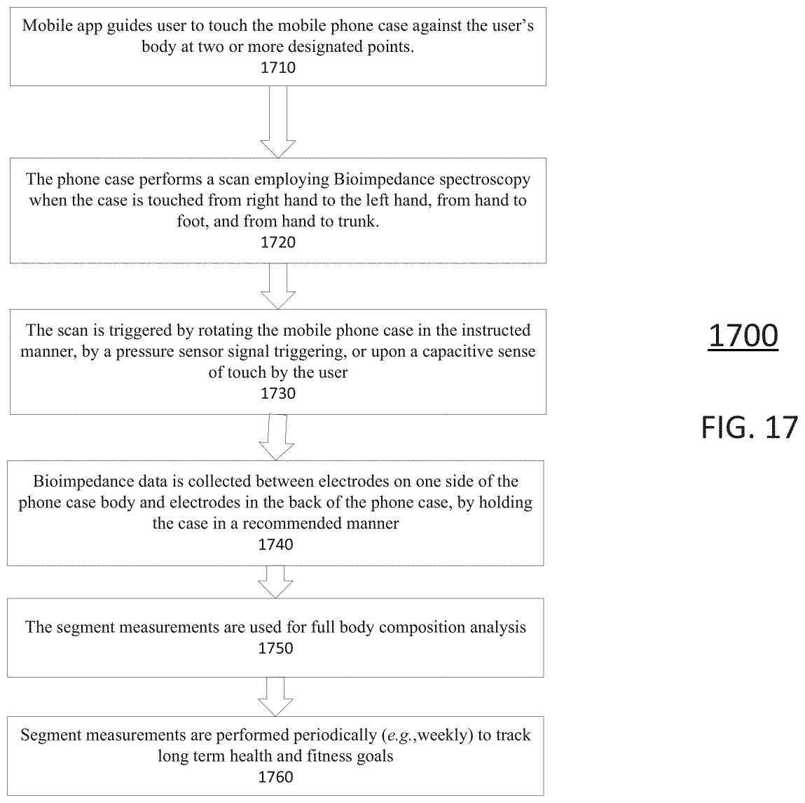

FIG. 17 illustrates a process for segment measurement, according to one embodiment. As depicted in FIG. 17, the mobile phone case performs segment analysis 1700. To take segment measurements, the user is guided by a mobile app to touch the mobile phone case against the user's body at two or more designated points at 1710. For instance, the phone case can perform a scan employing bioimpedance spectroscopy when the case is touched from right hand to the left hand, from hand to foot, and from hand to trunk at 1720. The scan can be triggered by rotating the mobile phone case in the instructed manner, by a pressure sensor signal triggering at, or upon a capacitive sense of touch by the user at 1730. Bioimpedance data is collected between electrodes on one side of the phone case body and electrodes in the back of the phone case, by holding the case in a recommended manner at 1740. The resulting segment measurements enable full body composition analysis at 1750. Segment measurements can be performed at a much larger interval, for example, on a weekly basis, for tracking long term health and fitness goals at 1760. These measurements are taken as often as a user feels convenient. This data is information-rich for health analysis.

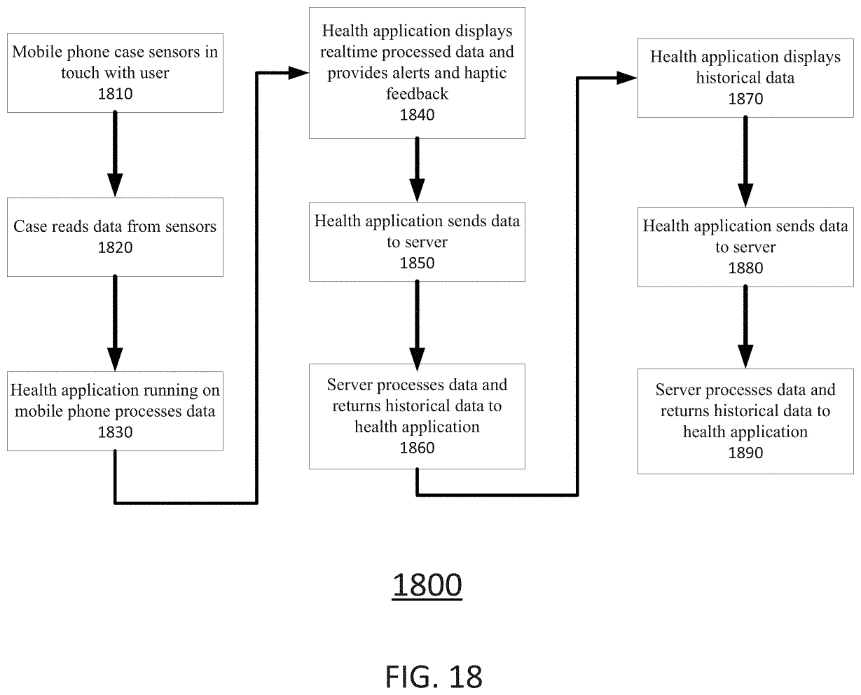

FIG. 18 illustrates a process for deep data analysis using the present mobile phone case, according to one embodiment. FIG. 18 depicts a data flow diagram for an embodiment of the system. The mobile phone case with sensors makes contact with the user at 1810 and the case reads data from those sensors at 1820. The health application running on the mobile phone processes and analyzes the data at 1830. The health application displays real-time processed data and provides alerts and haptic feedback to the user at 1840. In another embodiment, the health application sends the data to a central server at 1850. The central server processed the data and returns historical data to the health application at 1860. The health application displays the historical data at 1870. The health application sends the data to a server at 1880. The server processes the data and returns historical data to the health application at 1890.

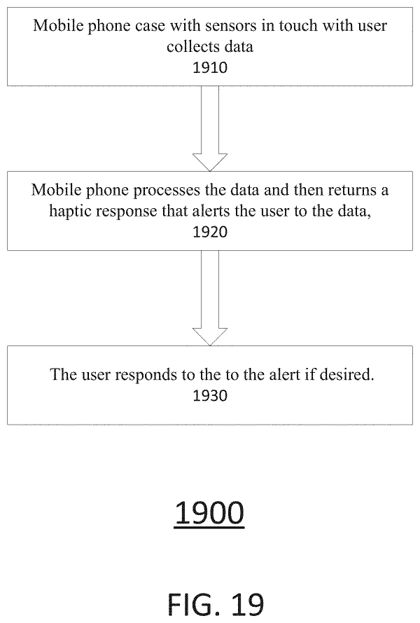

FIG. 19 illustrates a process for collecting data for haptic feedback, according to one embodiment. User data is collected by a mobile phone case upon user touch at 1910. The mobile phone processes the data and then returns a haptic response that alerts the user to the data at 1920. If desired, the user responds to the alert at 1930.

Although the various embodiments described herein, describe functionality performed by the present phone case, a person of skill in the art would understand that the functions described herein may be performed by a combination of mobile device, the present phone case, and servers. A person of skill in the art would also understand that the functions described herein may be performed by a mobile phone without the present phone case if the mobile phone includes one or more of the components described as part of the present phone case. Processing performed on the mobile device may also be performed on a server. A person of skill in the art would understand that the phone case may communicate with a mobile device through a standard communications port (e.g., USB, Lightning, etc.) or wirelessly (e.g., Bluetooth, WiFi, EHF, UHF, etc.).

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

What is claimed is:

1. A system comprising:
a case suitable for use with a mobile device; and
a first recess in the case, wherein the first recess has first optoelectronic sensors and first electrodes that facilitate one or more scans, wherein the scans include bioimpedance measurements and optical scans.

2. The system of claim 1, further comprising a second recess in the case, wherein the second recess has a second set of electrodes that facilitate the scans.

3. The system of claim 2, wherein the scans occur upon incidental touch with the case.

4. The system of claim 1, further comprising a capacitive touch sensor within the case.

5. The system of claim 4, wherein the first optoelectronic sensors activate upon incidental touch of the mobile device using a capacitive touch functionality of the capacitive touch sensor.

6. The system of claim 5, wherein a tap on the first recess initiates the one or more scans using the capacitive touch sensor.

7. The system of claim 1, further comprising tactile sensors to guide a finger for placement on the first recess in the case.

8. The system of claim 1, further comprising an extension unit for a scan.

9. The system of claim 8, further comprising an array of electrodes for spot measurement and impedance tomography using bioimpedance.

10. The system of claim 1, further comprising a food that is analyzed by the case.

11. The system of claim 1, wherein the system generates data for electrocardiographs.

12. The system of claim 1, wherein the system performs near-infrared spectroscopy for glucose level measurement and heart rate monitoring.

13. The system of claim 1, wherein the system performs incidental measurement each time a user holds the mobile device and enables long term health monitoring.

14. The system of claim 1, wherein the system further performs spot measurements.

15. The system of claim 14, wherein the spot measurements use an extension measurement cable.

16. The system of claim 1, wherein the system provides alerts for abnormal conditions, the abnormal conditions including one or more of diabetes, dehydration, hypertension, cardiovascular anomalies, breast cancer and prostate cancer.

17. The system of claim 1, wherein the case further comprises one or more of a pressure sensor, a moisture sensor, a temperature sensor, a humidity sensor, a galvanic skin resistance sensor, an infrared thermopile sensor, and a sweat analyzer.

18. The system of claim 1, wherein the first optoelectronic sensors are an LED array and a laser diode array.

19. The system of claim 1, further comprising a capacitive touch sensor within the mobile device to initiate the scans.

20. The system of claim 1, further comprising a server that collects data related to the scans and analyzes the data to identify abnormal conditions.
